SEQUENCE LISTING (16 sequences)

SEQ ID NO: 1 - protein, 74 aa, Bungarus caeruleus
LLCHTTSTSPISTVTCPSGENLCYTKMWCDAFCSSRGKVIELGCVATCPQPKPYEEVTCCSTDKCNPHPKQRPG

SEQ ID NO: 2 - protein, 73 aa, Bungarus multicinctus
IVCHTTATSPISAVTCPPGENLCYRKMWCDFCSSRGKVVELGCAATCPSKKPYEEVTCCSTDKCNPHPKQRPG

SEQ ID NO: 3 - protein, 72 aa, Dendroaspis polylepis
RTCNKTFSDQSKICPPGENICYTKTWCDAFCSQRGKRVELGCAATCPKVKAGVEIKCCSTDNCNKFQFGKPR

SEQ ID NO: 4 - protein, 71 aa, Naja kaouthia
IRCFITPDITSKDCPNGHVCYTKTWCDAFCSIRGKRVDLGCAATCPTVKTGVDIQCCSTDNCNPFPTRKRP

SEQ ID NO: 5 - protein, 71 aa, Naja nivea
IRCFITPDVTSQACPDGHVCYTKMWCDNFCGMRGKRVDLGCAATCPKVKPGVNIKCCSRDNCNPFPTRKRS

SEQ ID NO: 6 - protein, 72 aa, Bungarus multicinctus
CHTTATSPISAVTCPPGENLCYRKMWCDVFCSSRGKVVELGCAATCPSKKPYEEVTCCSTDKCNPHPKQRPG

SEQ ID NO: 7 - protein, 91 aa, synthetic construct
TKLEVVAATPTSLLISWDHPARYVIFYVITYGETGGNSPVQKFTVPGQKNTATISGLKPGVDYTITVYAMYFFWYRGRYVYGHPISINYRT

SEQ ID NO: 8 - protein, 72 aa, Dendroaspis polylepis
RTCNKTASDQSKICPPGENICYTKTWCDAFCSQRGKRVELGCAATCPKVKAGVEIKCCSTDNCNKFQFGKPR

SEQ ID NO: 9 - protein, 72 aa, Dendroaspis polylepis
RTCNKTFSDQSKICPPGENICYTKTWCDAACSQRGKRVELGCAATCPKVKAGVEIKCCSTDNCNKFQFGKPR

SEQ ID NO: 10 - protein, 72 aa, Dendroaspis polylepis
RTCNKTFSDQSKICPPGENICYTKTWCDAFCSQVGKRVELGCAATCPKVKAGVEIKCCSTDNCNKFQFGKPR

SEQ ID NO: 11 - protein, 72 aa, Dendroaspis polylepis
RTCNKTFSDQSKICPPGENICYTKTWCDAFCSQRGKVVELGCAATCPKVKAGVEIKCCSTDNCNKFQFGKPR

SEQ ID NO: 12 - protein, 72 aa, Dendroaspis polylepis
RTCNKTFSDQSKICPPGENICYTKTWCDAFCSQRGKRVELGCAATCPKVKAGVEIKCCSTDNCNKFQRGKPR

SEQ ID NO: 13 - protein, 74 aa, Bungarus multicinctus
IVCHTTATSPISAVTCPPGENLCYRKMWCDVFCSSRGKRVELGCAATCPSKKPYEEVTCCSTDKCNPHPKQRPG

SEQ ID NO: 14 - protein, 74 aa, Bungarus multicinctus
IVCHTTATSPISAVTCPPGENLCYRKMWCDVFCSSRGKRVELGCAATCPSKKPYEEVTCCSTDKCNPFPKQRPG

SEQ ID NO: 15 - protein, 72 aa, Bungarus multicinctus
RTCNKTFSDQSKICPPGENLCYRKMWCDVFCSSRGKRVELGCAATCPSKKPYEEVTCCSTDKCNPHPKQRPG

SEQ ID NO: 16 - protein, 72 aa, Dendroaspis polylepis
RTCNKTFSDQSKICPPGENICYTKTWCDAFCSQRGKRVELGCAATCPKVKAGVEIKCCSTDNCNPHPKQRPG

(19) United States
(12) Patent Application Publication
(10) Pub. No.: US 2025/0304665 A1
(43) Pub. Date: Oct. 2, 2025

(54) Systems and Methods for Protein Design Using Deep Generative Modeling

(71) Applicant: The Board of Trustees of the Leland Stanford Junior University, Stanford, CA (US)

(72) Inventors: Possu Huang, Stanford, CA (US); Raphael Eguchi, Stanford, CA (US); Christian A. Choe, La Mirada, CA (US)

(73) Assignee: The Board of Trustees of the Leland Stanford Junior University, Stanford, CA (US)

(21) Appl. No.: 18/865,674

(22) PCT Filed: May 15, 2023
(86) PCT No.: PCT/US2023/067019

§ 371 Date: Nov. 13, 2024

Related U.S. Application Data
(60) Provisional application No. 63/364,703, filed on May 13, 2022.

(51) Int. Cl. C07K 16/18; C12N 15/10; G01N 33/68; G16B 15/30; G16B 40/20

(52) U.S. Cl. CPC C07K 16/18; C12N 15/1037; G01N 33/6845; G16B 15/30; G16B 40/20; C07K 2317/569

(57) ABSTRACT

Systems and methods for determining molecular structures based on deep generative models an interaction field are described. Deep generative models can be utilized in combination with interaction field to design structures of molecules to target proteins, nucleic acids, and small molecules.

### CROSS-REFERENCE TO RELATED APPLICATIONS

The current application claims the benefit of and priority to U.S. Provisional Patent Application No. 63/364,703 entitled “Systems and Methods for Protein Design Using Deep Generative Modeling” filed May 13, 2022. The disclosure of U.S. Provisional Patent Application No. 63/364,703 is hereby incorporated by reference in its entirety for all purposes.

### SEQUENCE LISTING

The present invention contains a Sequence Listing which has been submitted electronically in ASCII format and is hereby incorporated by reference in its entirety. The ASCII copy was created on Jul. 31, 2023, is named 07887PCT.xml, and is 16,384 bytes in size.

### FIELD OF THE INVENTION

The present invention generally relates to systems and methods to design and synthesize proteins based on three dimensional structures; and more particularly to systems and methods that utilize deep generative modeling to determine the structures and sequences of synthesized proteins.

### BACKGROUND

Protein design and engineering can be helpful to the discovery effort of scientific industry, such as pharmaceuticals. Computational protein design has enabled the creation of a variety of de novo proteins. However, designing a protein of both structure and sequence that complement a target has been challenging. Current approaches employ screening massive random libraries, with little consideration towards the features of the target molecule. Advances in protein design would broaden its applications in the industrial innovation and development process.

### BRIEF SUMMARY

Systems and methods in accordance with various embodiments of the invention enable the design and/or synthesis of proteins based on structural and compositional properties. In many embodiments, proteins with specific structures and sequences can be synthesized for a wide range of product development processes such as drug discovery for the pharmaceutical industry, and material design for the agricultural and chemical industries.

One embodiment includes a method of synthesizing a binding protein comprising:
identifying a target structure;
constructing at least one interaction field describing physical interaction properties of atoms in the target structure;
optimizing a binding protein to the target structure:
   generating a candidate binding protein using a generative model;
   fitting the candidate binding protein and target structure in a virtual space according to a homogenous transformation function;
   using a loss function to produce an error value which evaluates a binding affinity between the candidate binding protein and the target structure based on the at least one interaction field; and
   while the error value is above a threshold value, providing the error value to the generative model and to the homogenous transformation function in order to inform the generation of a subsequent candidate binding protein and fitting to lower the error value;
outputting the binding protein until the error value reaches a stopping threshold; and
synthesizing the binding protein.

In another embodiment, the target structure is selected from the group consisting of: a protein, a region of a protein, an epitope, an antibody, a polypeptide, a region of a polypeptide, a nucleic acid, a DNA, an RNA, a sugar molecule, a monosaccharide, a disaccharide, a polysaccharide, and a small molecule.

In a further embodiment, the at least one interaction field comprises interactions selected from the group consisting of: Coulomb interactions, hydrogen bonds, π-π interactions, cation-π interactions, van der Waals interactions, and a virtual constraint.

An additional embodiment further comprises providing the generative model with a template backbone structure based on the target structure, wherein the template backbone structure is selected from the group consisting of: a monobody, an antibody, a nanobody, a single-chain variable fragment, a designed ankyrin repeat protein, and a lectin.

In a further yet embodiment, the generative model iteratively modifies the template backbone structure.

Another further embodiment comprises generating amino acid sequences of the binding protein.

Yet another embodiment further comprises ranking a set of the subsequent candidate binding proteins based on their binding affinity to the target structure.

In another additional embodiment, the synthesized binding protein is configured to be used in prokaryotes or eukaryotes.

In yet another embodiment again, the synthesized binding protein is configured to be used in in vitro or in vivo assays.

Another embodiment includes a method of synthesizing a binding protein comprising:
identifying a target structure;
generating at least one interaction field describing physical interaction properties of atoms in the target structure;
generating a candidate binding protein using a generative model;
fitting the candidate binding protein and the target structure in a virtual space according to a homogenous transformation function;
using a loss function to produce an error value which evaluates a binding affinity between the candidate binding protein and the target structure based on the at least one interaction field;
providing the error value to the generative model and to the homogenous transformation function;
generating a subsequent candidate binding protein using the generative model and the error value;
fitting the subsequent candidate binding protein and the target structure according to the homogenous transformation function and the error value; and
synthesizing the subsequent candidate binding protein as the binding protein.

In yet another embodiment, the target structure is selected from the group consisting of: a protein, a region of a protein, an epitope, an antibody, a polypeptide, a region of a polypeptide, a nucleic acid, a DNA, an RNA, a sugar molecule, a monosaccharide, a disaccharide, a polysaccharide, and a small molecule.

In an additional further embodiment, the at least one interaction field comprises interactions selected from the group consisting of: Coulomb interactions, hydrogen bonds, π-π interactions, cation-π interactions, van der Waals interactions, and a virtual constraint.

A further yet embodiment comprises providing the generative model with a template backbone structure based on the target structure, wherein the template backbone structure is selected from the group consisting of: a monobody, an antibody, a nanobody, a single-chain variable fragment, a designed ankyrin repeat protein, and a lectin.

In yet another embodiment, the generative model iteratively modifies the template backbone structure.

Another embodiment further comprises generating amino acid sequences of the subsequent candidate binding protein.

Yet another embodiment further comprises ranking a set of the subsequent candidate binding proteins based on their binding affinity to the target structure.

In a further embodiment again, the synthesized binding protein is configured to be used in prokaryotes or eukaryotes.

In another further yet embodiment, the synthesized binding protein is configured to be used in in vitro or in vivo assays.

An additional embodiment includes a method for generating a binding molecule, comprising:
identifying a target structure having a target binding site;
generating at least one interaction field describing physical interaction properties of atoms in the target binding site;
using a generative model to create a 3D model of a candidate binding molecule;
fitting the candidate binding molecule to the target binding site using a homogenous transformation function based on the at least one interaction field in a virtual space containing the 3D model of the candidate binding molecule and the target structure;
calculating an error in the fitting using a loss function; and
refining the candidate binding molecule and the homogenous transformation using the error until a stopping threshold is reached.

In a further yet embodiment, the stopping threshold is a predetermined number of iterations.

In another embodiment, the stopping threshold is a minimum acceptable error value.

In yet another embodiment, the stopping threshold is a minimum change in error value required to continue the refining.

In a further embodiment again, the target binding site is on a surface of the target structure.

In another further embodiment again, the loss function further determines a number of residues allowed to overlap the interaction field.

In a yet further embodiment, the target structure is selected from the group consisting of: a protein, a region of a protein, an epitope, an antibody, a polypeptide, a region of a polypeptide, a nucleic acid, a DNA, an RNA, a sugar molecule, a monosaccharide, a disaccharide, a polysaccharide, and a small molecule.

In another additional embodiment, the at least one interaction field comprises interactions selected from the group consisting of: Coulomb interactions, hydrogen bonds, π-π interactions, cation-π interactions, van der Waals interactions, and a virtual constraint.

In a further embodiment again, the 3D model is selected from the group consisting of: a monobody, an antibody, a nanobody, a single-chain variable fragment, a designed ankyrin repeat protein, and a lectin.

In yet another embodiment, the generative model iteratively modifies the 3D model.

A yet further embodiment, comprises ranking a set of the candidate binding molecules based on their binding affinity to the target structure.

Additional embodiments and features are set forth in part in the description that follows, and in part will become apparent to those skilled in the art upon examination of the specification or may be learned by the practice of the disclosure. A further understanding of the nature and advantages of the present disclosure may be realized by reference to the remaining portions of the specification and the drawings, which forms a part of this disclosure

### BRIEF DESCRIPTION OF THE DRAWINGS

The description will be more fully understood with reference to the following figures, which are presented as exemplary embodiments of the invention and should not be construed as a complete recitation of the scope of the invention. It should be noted that the patent or application file contains at least one drawing executed in color. Copies of this patent or patent application publication with color drawing(s) will be provided by the Office upon request and payment of the necessary fee.

FIG. 1 illustrates a process for synthesizing molecules in accordance with an embodiment of the invention.

FIG. 2 illustrates a process for synthesizing epitope specific proteins in accordance with an embodiment of the invention.

FIGS. 3A-3F illustrate performances of Sculptor components in accordance with an embodiment of the invention.

FIGS. 4A-4E illustrate recovery and redesign of a native complex in accordance with an embodiment of the invention.

FIG. 5 illustrates sculpting trajectories and blind recovery of designs in accordance with an embodiment of the invention.

FIGS. 6A-6D illustrate epitope mapping of a Sculptor-designed venom toxin binder in accordance with an embodiment of the invention. FIG. 6A includes SEQ ID NOs: 1-5.

FIGS. 7A-7B illustrate rescue of T2 binding using designed epitope features in accordance with an embodiment of the invention. FIG. 7A includes SEQ ID NOs: 3; 6.

FIG. 8A illustrates sequence for TGF-beta1 binder in accordance with an embodiment of the invention. FIG. 8A includes SEQ ID NO: 7.

FIG. 8B illustrates characterization of the TGF-beta1 binder interface in accordance with an embodiment of the invention.

FIG. 9 illustrates a heatmap of the result relative to wildtype enrichment in accordance with an embodiment of the invention.

FIGS. 10A-10B illustrate Shannon entropy in accordance with an embodiment of the invention.

FIG. 11 illustrates a protein structure based on the entropy calculation in accordance with an embodiment of the invention.

FIG. 12 illustrates yeast surface display of various binders analyzed with fluorescence assisted cell sorting in accordance with an embodiment of the invention.

FIG. 13 illustrates three-finger toxin sequences and mutated toxin sequences in accordance with an embodiment of the invention. FIG. 13 includes SEQ ID NOs: 1-5; 8-16.

### DETAILED DESCRIPTION

The interactions between proteins are critical to many biological processes. Many pharmaceuticals operate by specifically binding to target proteins in the body. Studies have shown that engineered proteins that target protein-protein interfaces can perform as effective therapeutics and modulators of cell signaling. Engineering binding proteins against specific target binding sites is a non-trivial task. Conventional processes have been limited by the difficulty of sampling flexible protein conformations and have focused on modifying the surface sequences of known protein scaffolds with limited backbone flexibility to model the interaction with a target site. Some methods reuse known protein interface contact residues and identify suitable protein scaffolds to host them. However, the creation of epitope-specific binders has remained difficult due to the complexity of the physiochemical environment for a chosen epitope and the need to create a stable protein with both a backbone and a sequence that will be compatible with the epitope.

Systems and methods described herein (referred to as “Sculptor”) can be used to automatically generate structural models and sequences for entire proteins and paratopes that are designed to target any arbitrarily provided epitope. In many embodiments, the generated sequences are iteratively altered in order to find increasingly better bindings. The generated protein can be altered in its entirety, including the backbone. In a variety of embodiments, the target is specifically an epitope of a given arbitrary protein. In numerous embodiments, via modifications discussed below, systems and methods described herein can generate protein and/or non-protein (such as, but not limited to, small molecules, nucleic acids, RNAs, DNAs, polysaccharides, monosaccharides, and disaccharides) binders against protein and/or non-protein epitopes that have similar binding effects. In numerous embodiments, a generative model is iteratively used to generate new proteins. As can readily be appreciated, while epitopes and paratopes refer to specific types of molecular binding, one skilled in the art will recognize that systems and methods described herein can be used to generate any type of binding molecule (e.g., with modifications discussed below) without departing from this invention.

In many embodiments, systems and methods described herein are provided with a target structure, e.g. a protein with a target epitope, and output a protein which will bind the epitope. The backbone can be automatically modified during an iterative generative process which enables the usage of significantly more binding conformations. In certain embodiments, interaction fields can be constructed which encode the way that the target structure interacts. In various embodiments, interaction fields include (but are not limited to) Coulomb interactions, hydrogen bonds, π-π, cation-IT, and van der Waals interactions. In various embodiments, other objective functions can be encoded into the interaction field, many of which can be defined virtually. Interaction fields can be constructed in different ways. In some embodiments, interaction fields can be constructed using a method that separates target definition from binder conformational sampling following an optimization method (e.g. latent space optimization, diffusion models, reinforcement learning, etc.). The field can use any part of the proteins or polymers in accordance with some embodiments. In some embodiments, the field in proteins can be constructed using sidechain-sidechain interactions, sidechain-backbone interactions, and/or backbone-backbone interactions. Several embodiments provide that the field can be constructed using contact pairs, knowledge-based potential, and/or neural nets to turn the field into a differentiable density function. Further, interaction fields can encode amino acid specific interactions if the binder scaffold is based on a protein, and/or other chemical moieties if based on other polymer types (such as, but not limited to, nucleic acids, DNAs, RNAs, polysaccharides, monosaccharides, disaccharides, small molecules, etc.). As can readily be appreciated, any number of different physical interactions can be encoded depending on the target type.

Once an interaction field is constructed, generative models in accordance with certain embodiments of the invention can create a candidate protein to bind the target. The generated protein and target can be matched in virtual space and transformed in the virtual space, where the binding between the two molecules can be evaluated. The error in the binding as measured by a loss function using the interaction field as described herein can then be provided to the generative model to inform the next iteration of the candidate protein. The error can further be provided to the transformation to better evaluate and refine/satisfy the physical orientation required by the binding. In many embodiments, the loss function produces multiple metrics stored as a vector which can be variously provided to the generative model as well as a homogenous transformation function. Over numerous iterations of this optimization process, better candidates are produced until a sufficiently good candidate is found. In certain embodiments, generatively designed molecules with specific structures can be synthesized. Synthesized molecules in accordance with various embodiments of the invention include (but are not limited to): proteins, mini-proteins, polypeptides, peptides, antibodies, monobodies, nanobodies, single-chain variable fragments (ScFv's), designed ankyrin repeat proteins (DARPins), lectins, other polymers and small molecules. Several embodiments apply the synthesized molecules in prokaryotes and/or eukaryotes. In various embodiments, designed molecules can be synthesized and evaluated using in vitro assays. In a number of embodiments, designed molecules can be used in in vitro assays.

Synthetic datasets can be used in the process of building generative models. Some embodiments use generative models to capture the general structural dynamics and conformational flexibility, which can be assisted by building Ig-VAE models with MD simulation augmented conformational ensembles. (See, e.g., R. R. Eguchi, et al., Ig-VAE: Generative Modeling of Protein Structure by Direct 3D Coordinate Generation. bioRxiv, 2022. Publisher: Cold Spring Harbor Laboratory; the disclosure of which is herein incorporated by reference.) Several embodiments use data created by IgFold for antibody generative models. (See, e.g., Graylab/lgFold at GitHub; the disclosure of which is herein incorporated by reference.) The structure ensemble used for training to capture the conformational flexibility can also be created by methods including (but not limited to) Rosetta software.

Several embodiments design molecules targeting a target including (but not limited to) proteins, regions of a protein, polypeptides, regions of a polypeptide, nucleic acids, regions of a nucleic acid, glycans, sugar moieties, polysaccharides, monosaccharides, disaccharides, general polymers, and/or small molecules. The target molecules and/or regions can be natural or synthetic. The target molecules and/or regions may have known structures. Several embodiments provide binding affinities of the synthesized molecules with the target in vitro and/or in vivo.

During optimization, the interacting residues on both the backbones and the target can be dynamically reassigned in accordance with many embodiments. The optimization can occur in real-time. In several embodiments, the optimization processes can be carried out using processes including (but not limited to) linear sum assignment to minimize fitting loss. Various embodiments incorporate dynamic loop assignment into Sculptor optimization loop for structurally variable CDR loops in antibody designs. In a variety of embodiments, joint optimization of the set of interacting residues, generative latent vector, and homogenous transformation parameters are achieved via gradient descent and Monte Carlo optimization. Some embodiments provide decision making processes including (1) fitting to the field; (2) making decisions to the number of residues that may be needed to overlap the field; (3) the generated structure may touch other regions outside of the defined epitope. Several embodiments design and optimize the amino acid sequences of the 3D structures. In some embodiments, the optimized 3D structures can be passed to a neural network-based sequence design module which can provide homology-informed sequences that are combined with field-specified residues to propose candidate amino acids at each position. In a number of embodiments, unrestricted interface optimization can be performed using protein modeling software, such as (but not limited to) Rosetta. In many embodiments, the sequence designs can be carried out using the generative models. Some embodiments design epitope-specific binders including (but not limited to) proteins, monobodies, antibodies, nanobodies, nucleic acids, DNAs, RNAs, polysaccharides, glycans, sugar polymers, small molecules etc. As noted above, simple modifications to the template 3D backbone, interaction field, and target binding site can be performed to generating binding molecules for any arbitrary target.

### Protein Design

Deep learning models have garnered great interest as general function approximators. Many deep learning models have become prominent tools in protein science. One application can be found in PSIPRED, which used neural networks to predict secondary structure from primary sequence. Deep learning models were also used for domain classification, protein-protein interaction mapping, and for contact map and distogram prediction in programs such as AlphaFold, trRosetta, RoseTTAFold, and RaptorX. Despite these applications, deep learning has seen little practical application in protein design. Although a collection of tools and potentially promising ideas have emerged, there do not yet exist working approaches to solve fundamental problems such as binding or catalysis. The vast majority of design algorithms have been sequence-based, making the creation of functional proteins difficult, which often require interaction with a secondary molecule.

Many essential biochemical processes and cell behaviors are regulated by protein-protein interactions (PPIs). Many studies have shown that engineered proteins targeting protein-protein-interfaces may serve as effective therapeutics, powerful modulators of cell signaling, and crucial components in recent CAR-T cell therapies. Despite both the demand and utility of epitope-specific binders, engineering these can be a challenge, with most methods requiring screening of massive random libraries, often with little consideration towards the features of the target epitope.

Computational protein design has enabled the creation of novel folds and a wide variety of de novo scaffolds. However, design of epitope-specific binders has remained difficult due to the need to create a foldable protein with both backbone and sequence that complement the epitope of interest. Results include RifDock that creates protein-protein binders. RifDock docks a collection of pre-built backbones into a rotamer interaction field, and is improved by iteratively enriching promising backbone motifs. (See, e.g., L. X. Cao, et al., Design of protein binding proteins from target structure alone, Nature, 2022; the disclosure of which is herein incorporated by reference.) Polizzi et. al. reported a conceptually similar method called COMBs, which designs helical bundles to bind to small molecules using a protein interaction field built around chemical groups. (See, e.g., N. Anand, Nature Communications, 13 (1): 746, 2022; the disclosure of which is herein incorporated by reference.) Similar to RifDock, the COMBs approach uses interaction units called “van der Mers” to identify valid backbone geometries from a set of pre-constructed helical bundles. While interaction field methods may be powerful, they depend on the creation of a sufficiently large backbone library to recover field-compatible structures, and little is understood about how to build such libraries or their compatibility with various epitopes. Though successful for some targets, the massive scope of the protein structural space may suggest that sampling a fixed number of backbones a priori may not be a generalizable approach to binder design-rigid backbones simply may not be able to fit certain interaction fields. Eguchi et al. has reported generative design of proteins using 3D coordinates. (See, e.g., R. R. Eguchi, et al., Ig-VAE: Generative Modeling of Protein Structure by Direct 3D Coordinate Generation. bioRxiv, 2022. Publisher: Cold Spring Harbor Laboratory; the disclosure of which is herein incorporated by reference.)

Modeling 3D structures can be important to designing functional proteins. While several algorithms for generating structures may exist, for example via a backbone-energy function or by neural network “hallucination”, these methods may not easily allow for conditioning on an arbitrary interacting partner-a feature important to designing functions such as binding.

Instead of relying on pre-constructed backbones, systems and methods of Sculptor in accordance with many embodiments design proteins for various targets without knowing the primary structures and/or the amino acid sequences of the protein. Several embodiments generate three-dimensional (3D) backbones of the protein based on the target. Certain embodiments apply interaction field including (but not limited to) amino acid specific interaction field to further construct and optimize the 3D backbones. Examples of interaction fields include (but are not limited to) Coulomb interactions, hydrogen bonds, π-π, cation-π, and van der Waals interactions. During optimization, the interacting residues on both the backbones and the target can be dynamically reassigned. The optimization occurs in real-time. In several embodiments, the optimization processes can be carried out using processes including (but not limited to) linear sum assignment to minimize fitting loss to optimize the 3D structure. In some embodiments, the optimized 3D structures can be passed to a neural network-based sequence design module which can provide homology-informed sequences that are combined with field-specified residues to propose candidate amino acids at each position. The amino acid sequences of the 3D structures can be optimized. In a number of embodiments, unrestricted interface optimization can be performed using Rosetta.

Many embodiments implement Sculptor that combines deep generative modeling and interactive field to create epitope-specific binders including (but not limited to) proteins, monobodies, antibodies, and nanobodies. The Sculptor algorithm in accordance with several embodiments include extensive searches over the positions, interactions, and generated conformations of a fold, and craft backbones to complement a user-specified epitope. Sculptor can be both modular and general since the generative model can be trained on any fold, allowing for scaffold choices such as monobodies, antibodies, nanobodies, ScFv's, DARPins, and lectins. Some embodiments design sequences onto the backbone using information from residue-wise interaction databases, convolutional sequence design modules, and Rosetta software.

Several embodiments are able to generate a binder against the desired epitope and achieve pan-binding across multiple venom toxins. Certain embodiments use Sculptor to design binders against a conserved epitope on venom toxins that is implicated in neuromuscular paralysis, and obtain a pan-toxin binder from a small library. Some embodiments use a small library of about 5800 designs-far smaller than conventional yeast display libraries which are often about 10⁷ sequences or more. A number of embodiments provide Sculptor may create broadly neutralizing binders. The generated proteins can be synthesized and experimentally-validated.

Systems and methods for synthesizing proteins with specific structures and sequences that can be generated by Sculptor in accordance with various embodiments of the invention are discussed further below.

### Sculptor

Many embodiments utilize Sculptor processes to design protein structures using deep generative modeling combined with interaction field. In order to design a binding protein that is complementary to the target interface at both the sequence and backbone structure level, four spaces are considered during design: (1) the space of rotational and translational degrees of freedom, (2) the space of backbone structure, (3) the space of interface assignments, and (4) the space of amino acid sequences. Sculptor algorithm in accordance with many embodiments focuses on joint optimization over spaces (1) to (3), while (4) can be searched using a combination of a learned sequence design module and Rosetta interface optimization after a backbone is created.

Several embodiments provide that the input to Sculptor can be a protein structure and a user-specified epitope. An interaction field can be constructed around the epitope using an amino-acid specific interaction database consisting of pairwise interactions in accordance with some embodiments. Some embodiments use a protein variational autoencoder (VAE) to define a massive space of possible binder conformations sampled using molecular dynamics simulations. Several embodiments use synthetic datasets to assist building generative models, such as IgFold for antibody generative models. In many embodiments, given a target field, the algorithm can randomly initialize the latent vector of a coordinate-generating VAE that defines the backbone conformational search space. Certain embodiments provide that homogenous transformation parameters that dictate the position of the binder in 3D space, and an assignment of interacting residue pairs between the target and binder can also be initialized. The core loop of Sculptor in accordance with some embodiments can jointly optimize all of these parameters to maximize interaction field fit using a combination of (but not limited to) gradient descent, Metropolis-Hastings, and linear sum assignment. Over the span of the trajectory, the initialized binder can be molded against the target interface as it is fitted into the interaction field in accordance with several embodiments. Once the protein backbone is optimized, sequence design in accordance with certain embodiments can be done using a convolution-based sequence design module in combination with Rosetta software. Residue choices from both the interaction field and the design module can be passed to Rosetta software which builds explicit side-chains and searches for jointly compatible sequences. In a final design step, the Rosetta software may be allowed to freely redesign the sculpted interface to optimize interactions. Various algorithms can be used to rank the designs before proceeding with synthesizing the proteins.

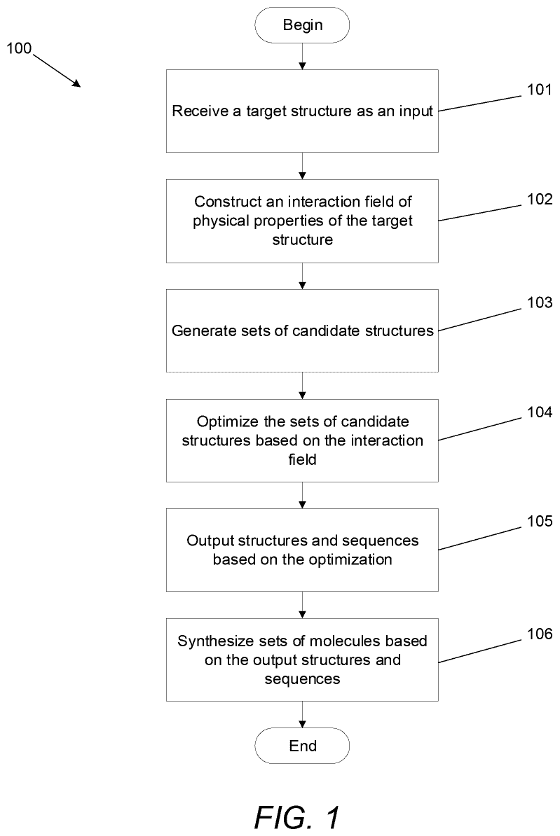

A process for synthesizing molecules in accordance with an embodiment of the invention is illustrated in FIG. 1. Process 100 receives (101) a target structure as an input. The target structures can be any types of a protein (such as an antigen), regions of a protein (such as a protein with a target epitope), polypeptides, regions of a polypeptide, and/or non-protein molecules (such as small molecules, nucleic acids, RNAs, DNAs, polysaccharides, monosaccharides, disaccharides, glycans, and sugar polymers). The target molecules and/or regions can be natural or synthetic. The target molecules and/or regions may have known structures. In a number of embodiments, the target is specifically an epitope of a given arbitrary protein.

Construct (102) an interaction field which encodes various physical interactions that the target structure interacts. Various objective functions can be encoded into the interaction field. The interaction field can encode amino acid specific interactions if the binder scaffold is based on a protein, and/or other chemical moieties if based on other polymer types (such as nucleic acids, DNAs, RNAs, polysaccharides, monosaccharides, disaccharides, glycans, sugar polymers, small molecules, etc.). Interaction fields in accordance with many embodiments of the invention can use any part of the proteins or polymers. In some embodiments, interaction fields in proteins can be constructed using sidechain-sidechain, sidechain-backbone, and/or backbone-backbone interactions. In certain embodiments, interaction fields in nucleic acids can be constructed using interactions between the nucleic acid bases and/or backbones. Interaction fields can include (but are not limited to) Coulomb interactions, hydrogen bonds, π-π, cation-π, and van der Waals interactions. Some interaction fields can be defined virtually. In several embodiments, interaction fields can be constructed using contact pairs, knowledge-based potential, neural nets to turn the field into a differentiable density function. A field can be constructed in different ways. In some embodiments, interaction fields can be constructed using a method that separates target definition from binder conformational sampling following an optimization method (such as, but not limited to, latent space optimization, diffusion models, reinforcement learning).

Generate (103) sets of candidate structures to bind the target using generative models once the interaction field is constructed. Template 3D backbone structures including (but not limited to) monobody, antibody, nanobody, single-chain variable fragment, designed ankyrin repeat protein, and lectin, can be provided to the generative models. Template 3D backbone structures can be selected based on the target structure. Certain embodiments use monobody as template 3D backbone for the generative models when the target is an epitope. Some embodiments use antibody as template 3D backbone for the generative models when the target is an antigen.

Optimize (104) the sets of candidate structures based on the interaction fields. The generated candidate structures (such as protein structures) and the target structure can be matched in virtual space and transformed in the virtual space, where the binding between the two molecules can be evaluated. The error in the binding can be measured by a loss function using interaction fields. The error can then be provided to the generative models to inform the next iteration of the candidate protein. The error can further be provided to the transformation to better evaluate, refine, and satisfy the physical orientation required by the binding. In many embodiments, the loss function produces multiple metrics stored as a vector which can be variously provided to the generative model as well as a homogenous transformation function. The optimization process (loop) can be repeated as many times as needed until a sufficiently good candidate is found. The optimized candidate structures have better binding affinity with the target structure.

Synthetic datasets can be used for building generative models. Some generative models capture the general structural dynamics and conformational flexibility, which can be assisted by building Ig-VAE models with molecular dynamics simulation augmented conformational ensembles. Generative models for antibody designs can use data created by IgFold. The structure ensemble used for training to capture the conformational flexibility can be created by various methods including (but not limited to) Rosetta software.

During optimization, the interacting residues on the backbones of the candidate proteins and the target can be dynamically reassigned. The optimization can occur in real-time. In several embodiments, the optimization processes can be carried out using processes including (but not limited to) linear sum assignment to minimize fitting loss. In a variety of embodiments, joint optimization of the set of interacting residues, generative latent vector, and homogenous transformation parameters can be achieved via gradient descent and Monte Carlo optimization.

Some embodiments incorporate dynamic loop assignment for variable complementary determining region (CDR) loops in antibody designs. Dynamic loop assignment occurs with each loop of the algorithm. At the start of each loop, the residues are changed in order to interact with the target epitope. Various methods such as a neural network classifier can be used to predict which residues are CDR loop residues. Once the CDR residues are identified, the interactions are restricted to be only with those residues. It is important to restrict the interactions with CDR residues, so the antibody binders interact with the target epitope via the CDR loops and not elsewhere in the protein.

Output (105) the optimized 3D structures and/or sequences of such structures. For candidate proteins, the protein structures and their amino acid sequences can be generated. For nucleic acids, the structures and the nucleic acid sequences can be generated. Optimized 3D structures can be passed to a neural network-based sequence design module which can provide homology-informed sequences that are combined with field-specified residues to propose candidate amino acids at each position. In a number of embodiments, unrestricted interface optimization can be performed using protein modeling software, such as (but not limited to) Rosetta. In many embodiments, the sequence designs can be carried out using the generative models. In certain embodiments, the output structures can be ranked based on the binding affinity using various software and/or algorithm such as Rosetta, AlphaFold, AlphaFold2, and AlphaFold-Colab.

Several embodiments synthesize (106) the output structures for various applications. The synthesized molecules can include (but are not limited to): proteins, mini-proteins, polypeptides, peptides, antibodies, monobodies, nanobodies, single-chain variable fragments (ScFv's), designed ankyrin repeat proteins (DARPins), lectins, other polymers and small molecules. The synthesized molecules can be applied in prokaryotes and/or eukaryotes for various applications. In various embodiments, designed molecules can be synthesized and evaluated using in vitro assays. In a number of embodiments, designed molecules can be used in in vitro assays. The binding affinities of the synthesized molecules with the target can be determined in vitro and/or in vivo.

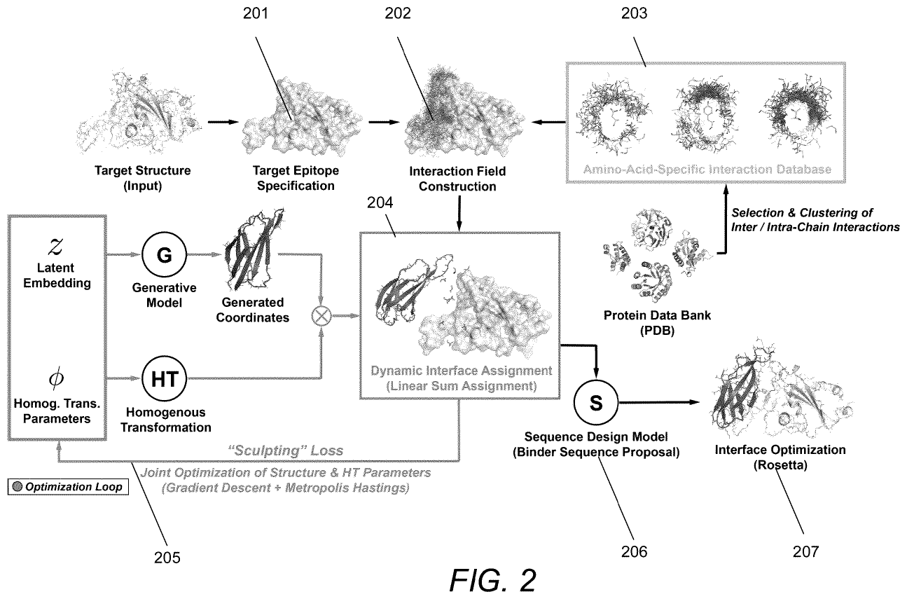

A scheme of the Sculptor algorithm in accordance with an embodiment is shown in FIG. 2. The input to sculptor can be a target structure with a user-specified epitope 201. An amino-acid-specific interaction field is built around the target epitope 202 using a database of clustered protein interactions harvested from protein data bank (PDB) 203. To perform interface design, a generated structure can be docked into the interaction field while jointly optimizing the binder backbone conformation and position 204. During optimization the interacting residues on both the binder and target can be dynamically reassigned via linear sum assignment to minimize fitting loss 205. Post-sculpting, the optimized structure is passed to a neural network-based sequence design module which provides homology-informed sequences that are combined with field-specified residues to propose candidate amino acids at each position 206. Final unrestricted interface optimization can be performed using Rosetta 207.

While various processes for designing and synthesizing proteins are described above with reference to FIG. 1 and FIG. 2, any of a variety of processes that utilize machine learning to generate the structures of proteins can be utilized in the design and/or synthesis of molecules as appropriate to the requirements of specific applications in accordance with various embodiments of the invention. Processes for deep generative designs of backbone structures in accordance with various embodiments of the invention are discussed further below.

### Deep Generative Model of Backbone Structures

In many embodiments, Sculptor processes perform conformational search space for backbone designs. A component of Sculptor is a deep variational autoencoder and provides a conformational search space for the design algorithm. In many embodiments, this model generates 3D coordinates that can be fully differentiable with respect to the latent vector, allowing for computation of gradients and structural optimization via latent space search.

Several embodiments may use monobody scaffolds as a design template. Monobodies are a family of binding proteins based on a modified fibronectin-3 scaffold, and have many publicly available high-resolution structures in complex with a wide range of targets. Monobodies can have similar length and a conserved core, but may have diverse loop sequences and structures. These features can be well-suited to sequence design module and generative model training because most variance in the data can be directly relevant to target specificity. To train the generative model, several embodiments select about 34 monobody PDBs of similar length and structurally pad these to be length 91 using Rosetta Remodel. Some embodiments randomly generate about 12 triple-mutants per structure, yielding a total of about 442 seed structures. To create the training datasets, several embodiments simulate the seed structures using molecular dynamics (MD). Frames from the simulation can be clustered and used to create a training set of about 1.6 M structures, yielding a dense conformational dataset biased towards native structures and augmented with physically realistic structures.

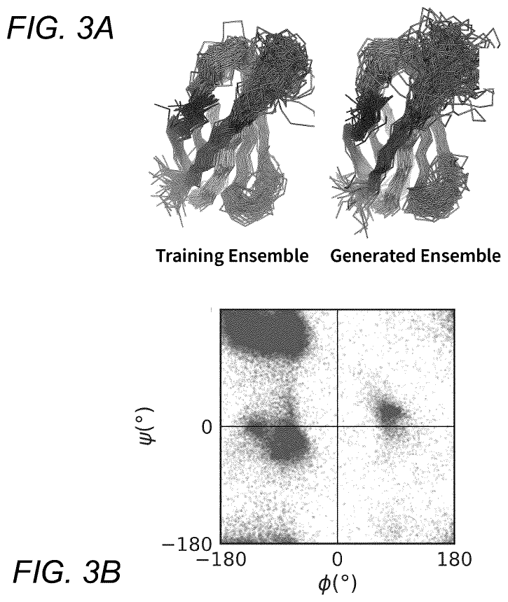

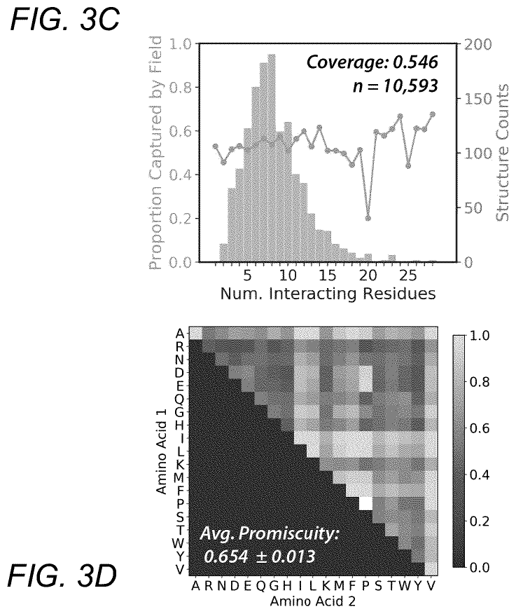

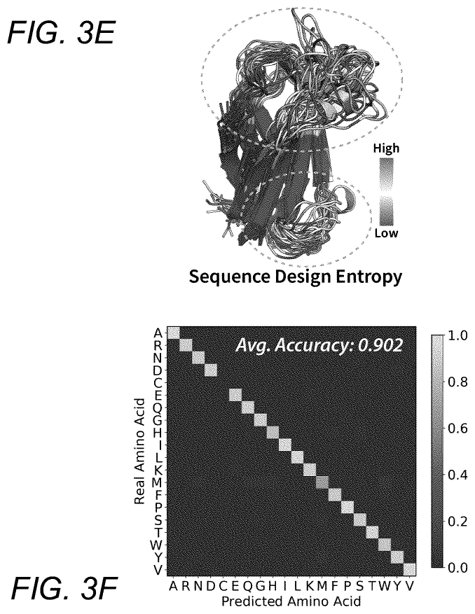

FIG. 3A illustrates comparison of about 100 randomly selected training set structures and 100 random VAE-generated structures in accordance with an embodiment. FIG. 3B illustrates comparison of Ramachandran distributions of the training and generated structures in accordance with an embodiment. FIGS. 3A and 3B analyze the performance of the generative model, showing backbone and torsional distributions of the training set and generated monobody structures. While slightly higher in variance, the generated structures are high in quality with the expected conformational and torsional propensities. VAE can adequately capture monobody conformational space.

### Pairwise Residue Interaction Field

Many embodiments implement interaction field constructed from amino acid interactions. Several embodiments provide analysis of the interaction database, which can be used to construct an interaction field from a user-specified epitope. These interactions can be harvested from the PDB using Arpeggio (See, e.g., H. C. Jubb, et al., Journal of molecular biology, 429 (3): 365-371, 2017; the disclosure of which is herein incorporated by reference.), and clustered via hierarchical clustering. The database includes both inter-chain and intra-chain polar and hydrogen bonding interactions. Assessment of field coverage using the PPI4DOCK benchmark set in accordance with an embodiment is illustrated in FIG. 3C. PPI4Dock contains about 1,417 complexes with about 10,593 amino acid interactions detected by Arpeggio. The 1,417 complexes are widely used to benchmark protein-protein docking algorithms. The proportion of residues captured by the interaction field versus the number of interacting residues in a complex is plotted in blue with standard errors. The coverage threshold can be defined as having backbone atom (N, Ca, C, O, Cb) alignment within about 0.5 Å dRMSD. The distribution of the numbers of interacting residues per complex is shown as a red histogram, and the percentage of interacting residues captured by the interaction field in blue. Overall the interaction field exhibits a coverage of about 55%, and this value remains relatively constant across different interface sizes. Some embodiments provide that this field coverage value does not indicate field quality, but rather that the design space can be comprised of the 55% most enriched interactions in PDB.

Promiscuity matrix of the interaction field in accordance with an embodiment is illustrated in FIG. 3D. Each matrix position may correspond to a pair of amino acid types, and the entry can be the proportion of interactions for which there is a similar interaction within about 0.5 Å dRMSD formed by other type-pairs. dRMSD can be computed for backbone atoms (N, Ca, C, O, Cb) between the two interacting residues. Each entry in this matrix indicates the proportion of interactions for a given amino acid pair that are geometrically similar to any other amino acid pair with different identities. This can be referred to as the promiscuity value. For example, for the pair (V,V) the entry value is close to 1.0. This indicates that nearly all (V,V) interactions have some non-(V,V) pair in the database that is very similar in relative positioning, which can be defined as having backbone atom alignment within 0.5 Å dRMSD. Notably, many residue pairs had a high promiscuity, with an overall average of about 65.4%. This may suggest that many residue-residue interactions adopt highly similar 3D positions independent of residue identity. This observation motivates the final step after sequence design, where Rosetta is allowed to freely redesign the interaction interface, since many field-specified pairs may have alternate amino acid identities that form favorable interactions.

### 3D-Convolutional Sequence Design Module

Several embodiments construct amino acid sequences after sculpting the backbone structures. Sculptor's sequence design scheme can be built with the intent to restrict the sequence search space by biasing designs towards known monobody sequences while also incorporating interaction-field-specified amino acid identities. Post backbone-sculpting, some embodiments provide 3D-convolution-based neural networks to perform sequence design. The design module can be trained as a classifier that takes a voxelized residue environment as input, and outputs a probability distribution over amino acid identity. Several embodiments provide that Sculptor's design module may use only backbone atoms to make side-chain-independent predictions at each position. Such design processes in accordance with certain embodiments may allow the model to capture backbone-position-dependent homology specific to the monobody fold, rather than being a general protein design tool. In many embodiments, the design module can be used to specify the sequence of the binder and does not “see” any features on the target epitope—these are instead handled by the interaction field. The outputs of the design module can be used to create a preliminary list of candidate amino acids at each position by selecting residue types above a fixed probability threshold in accordance with some embodiments. These candidates can be combined with the field-specified interface residues before being passed to Rosetta for joint design and repacking at all positions. By structuring the sequence design scheme this way, field-specified positions not used in the final interface design are biased towards a native-like distribution.

Position-wise entropy of sequence-design-module-predicted amino acid distributions for 30 generated backbones in accordance with an embodiment is illustrated in FIG. 3E. The residue-wise Shannon entropies from the sequence design module for 30 randomly generated backbones are shown. Monobody binding loops are circled in dash. The entropy of the amino acid distributions can be lower along core beta-strands and reflect the conserved sequence identities found in existing monobodies. Entropies can be noticeably higher in the two side-loops, which are used to bind a variety of targets and have the most variable sequences. The design module can correctly capture uncertainty in monobody sequence space and its backbone-positional dependence.

Test set confusion matrix for the deep sequence design module in accordance with an embodiment is illustrated in FIG. 3F. Design module accuracy and a confusion matrix are shown. Each amino acid type can be represented by 100 randomly selected examples, with the exception of cysteine, which are not present in native monobodies. Counts can be normalized horizontally by the number of real examples in each class. The module can achieve an average accuracy of about 90.2% over a balanced test set of 100 examples per amino acid type. Confusion between different residue types may be low (<0.2) overall.

### Recovery and Redesign of a Native Complex

Many embodiments provide functionalities of Sculptor design processes. Several embodiments provide recovery of the native structure of a monobody in an existing complex. As a test case, PDB:2OCF (A) can be selected, which is the structure of a monobody bound to the human estrogen receptor ligand-binding domain. This complex is selected because it uses a helical binding loop, which can be rare among known monobodies, making it both underrepresented in the training dataset and more difficult to recover via latent space search.

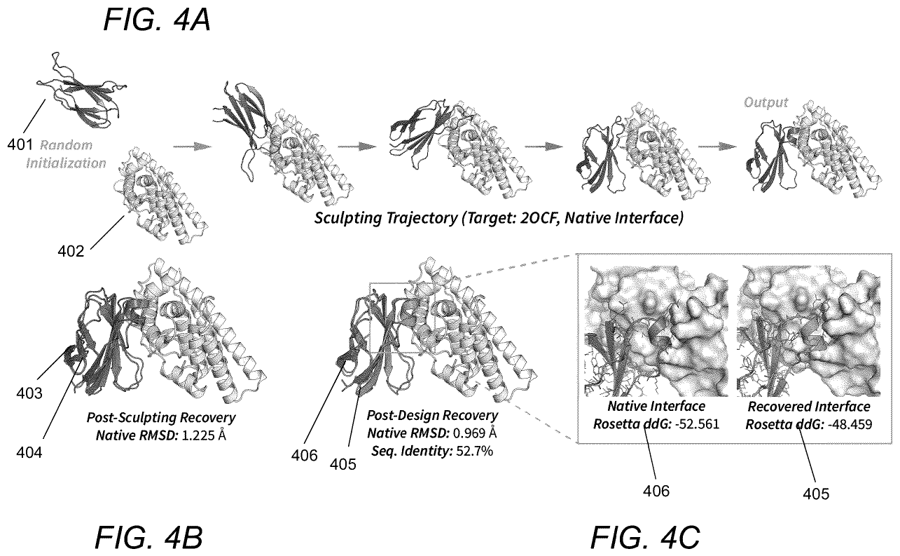

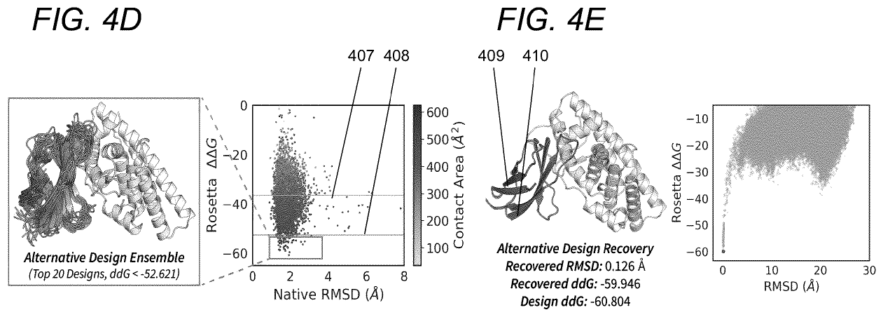

To test recovery of the 2OCF monobody, some embodiments specify the native epitope as the target interface and sample about 5,864 sculpting trajectories each initialized with a random backbone conformation and random position centered around the target protein. Sculpting trajectory against 2OCF (A) given the native interface assignment in accordance with an embodiment is illustrated in FIG. 4A. The generated molecule 401 and the target 402 are shown. Alignment of the post-sculpting structure in accordance with an embodiment is illustrated in FIG. 4B. The structure 403 and the native complex 404 are shown. Alignment of the post-sequence-design structure in accordance with an embodiment is illustrated in FIG. 4C. The structure 405 and the native complex 406 are shown. The insert depicts the full-atom rendering of the helical loop from each structure interacting with the target surface. FIGS. 4A and 4B show the trajectory that recovered the native complex most closely, and achieves a native RMSD of about 1.2 Å. Inspection of the trajectory suggests the algorithm traverses a large span of conformational space before it is molded against the target epitope and shaped into a helical structure (FIG. 4A). After sequence design and unconstrained refinement with Rosetta, native RMSD improves to about 0.97 Å. Notably, pre- and post-refinement outputs exhibit very little change in backbone structure (<1 Å RMSD), suggesting the complex recovered by Sculptor likely corresponds to a local energy minimum over the conformational and positional space explored by the algorithm. Post-design, the sequence identity of the recovered binder is about 52.7% relative to native. While sequence recovery may be low, the ddG of recovered and native sequences can be both approximately-50 Rosetta Energy Units (REU), indicating that the alternative sequence may be favored similarly to native.

Some embodiments generate novel binder designs that may differ from the native binder given the same epitope. Plot of ddG's of designs generated with the native 2OCF interface assignment in accordance with an embodiment is illustrated in FIG. 4D. FIG. 4D shows an ensemble of the top 20 designs ranked by ddG and show a plot of ddG versus native RMSD. The red line is drawn at the ddG of the native complex (−52.621 REU) and the blue line is drawn at the average ddG of all complexes in the PPI4DOCK dataset (−36.437 REU, n=1,417). The insert depicts the ensemble of top 20 designs with ddG values better than the native complex. Among the same set of 5,864 trajectories, Sculptor generates about 2,977 interfaces with ddG's better than the average ddG of PPI4DOCK (FIG. 4D, 407), among which 68 interfaces achieve ddG's better than the native 2OCF interface (FIG. 4D, 408). To further test epitope specificity, some embodiments provide blind global docking of the best scoring complex with RosettaDock. FIG. 4E illustrates docking recovery of the top alternative design from the ensemble shown in panel D in accordance with an embodiment. The designed structure is shown in 409 and the lowest ddG structure from the docking trajectory is shown in 410. The full trajectory (n=100,000 decoys) is shown in the plot on the right. The lowest ddG structure is shown as a red point. The designed complex can be recovered to within 1 Å RMSD, and the docking trajectory forms a robust funnel converging to a global energy minimum over 100,000 decoys. Several embodiments provide that Sculptor algorithm is able to recover the backbone structure of a native complex via the sculpting process, even when the loop structure is poorly represented. Further, Sculptor is able to generate designs that differ from known native binders, but are comparable in interface quality under Rosetta evaluation.

### Computational Validation of Epitope-Specific Designs

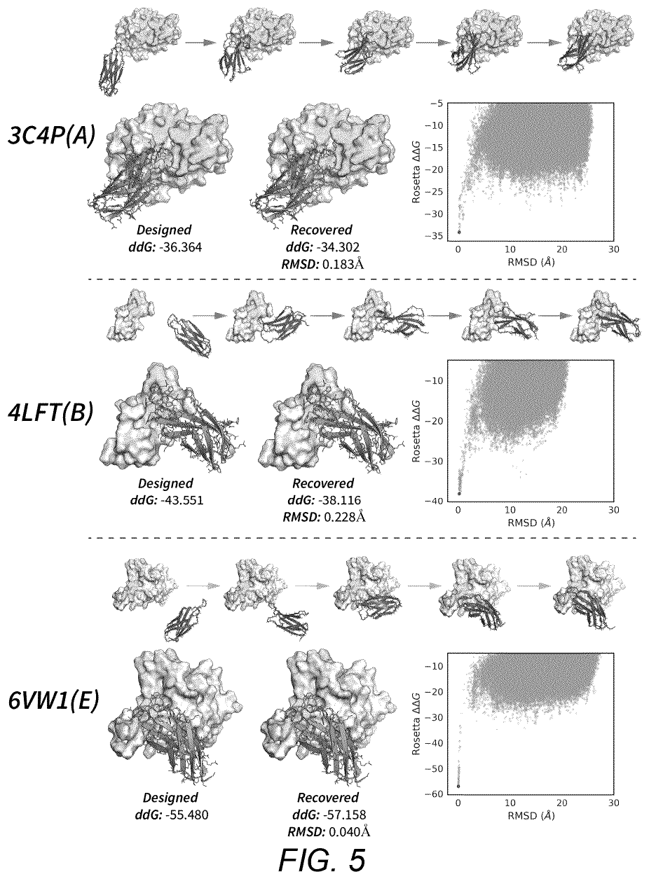

Many embodiments provide computational validations of epitope-specific binder designs. To assess algorithm performance against epitopes for which monobody binders do not exist, some embodiments select 3 targets: SHV-1 beta-lactamase PDB:3C4P (A), alpha-elapitoxin PDB:4LFT (B), and SARS-COV2-RBD PDB:6VW1 (E). For each of these targets, epitopes identified in literature as being biologically relevant can be chosen. The beta-lactamase epitope corresponds to a known inhibitory site, the toxin epitope corresponds to a motif on long-chain snake venom toxins needed for nicotinic acetylcholine receptor (nAChR) binding, and the RBD epitope corresponds to the ACE2 binding site. Sculpting trajectories and blind recovery of designs in accordance with an embodiment is illustrated in FIG. 5. The top sequence of structures depicts frames from the sculpting trajectory for each target starting from a random initialization. Targets with highlighted epitopes are shown. The structures correspond to the full-atom designs are shown in left. The structures on right are the structures recovered from blind global docking using RosettaDock. The docking trajectories are shown in right-side plots, with each run containing 100,000 decoys and minimum energy decoy shown as a dot. Sculptor creates about 3,000 designs against each epitope, and selects structures with the best ddG's from each set. In each trajectory the monobody structure can be initialized at a random position without any contacts to the target epitope before undergoing sculpting. In FIG. 5, blind global Rosetta docking can be used to computationally verify design specificity. All three examples closely recover the designed interface with docking trajectories forming robust funnels, and design binding modes constituting global energy minima over 100,000 decoys. Similar to the 2OCF designs, post-sculpting backbones may move very little after unconstrained design (<1 Å RMSD), suggesting again that post-sculpting designs appear adopt a local energy minimum under Rosetta (FIG. 4, Trajectory vs Designed).

While the sculpting process constitutes a search over possible interaction modes within the designated epitope, potential binders can be allowed to form any number of interactions and may not be required to mask the entire epitope. This flexibility allows the algorithm to adapt when the user specifies a very difficult-to-reach epitope. For example, by using only a small portion of a binding loop for interaction. This effect can be observable in differences in epitope coverage between the three examples, where Sculptor places a loop in a groove on the surface of 3C4P (A), while leaving the right side more exposed. In contrast, the designs against 4LFT (B) and 6VW1 (E) cover a larger proportion of the specified surface. This behavior can be controlled by the user by simply adjusting the size of the specified epitope. Overall the data suggest that Sculptor is able to generate realistic binders against a variety of epitopes that are favorable under computational benchmarks. In addition, the interface-search functionality of the algorithm is robust, as it is able to recover promising binding modes from user-specified epitopes even when the specified area is large.

### Design and Characterization of a Pan-Toxin Binder

While Sculptor exhibits promising behavior under computational benchmarks, many embodiments provide the design and synthesis of a biologically significant and challenging epitope. Several embodiments provide a conserved epitope on long-chain three-finger neurotoxins in snake venoms as a target, implicated in their ability induce skeletal muscle paralysis via postsynaptic nAChR's. Some embodiments use Sculptor to create a pan-neutralizing binder that could be used to recognize this epitope across multiple venom toxins.

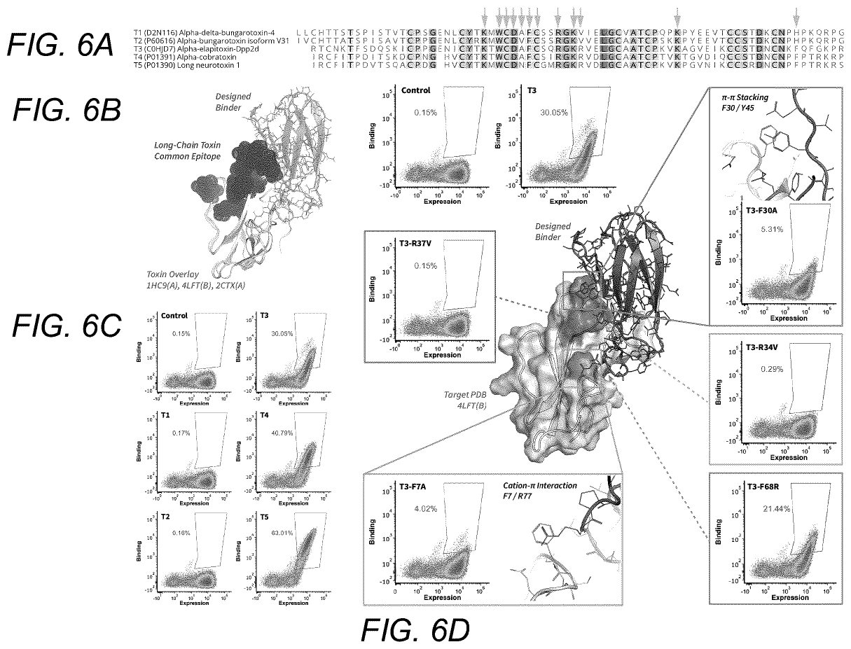

Five toxin variants which contained the conserved epitope can be selected as target sequences, labelling them T1 through T5. Sequence alignments of the five venom-toxin targets with gene accession numbers shown in parentheses is illustrated in FIG. 6A. The target epitope is indicated by red arrows. Crystal structures for T2, T3, and T4 are available on PDB and can be used as inputs to sculptor. The structural overlay of these targets is shown in FIG. 6B, with structures shown in white and the conserved motif shown in red. The overlaid PDBs correspond to T2, T3 and T4 respectively. For each of the 3 targets, about 8000 backbones can be sculpted, and designed sequences on backbones that fitted four or more field-interactions. The top 2000 binders for each target can be selected using a combined ranking of Rosetta ddG, Sc, and interface-buried-SASA. This may yield a total library size of about 5,923 designs against the common epitope. The full library can be transformed into yeast and screened against all five of the targets. Several embodiments are able to select and enrich for one binder designed against T3, and find that it bound to 3 of the 5 targets, specifically T3, T4 and T5, but did not bind to T1 or T2 (FIG. 6B). The structure of the designed binder is aligned against the conserved epitope in FIG. 6A. The binding profiles of the designed binder to the five targets are illustrated in FIG. 6C. The designed complex in accordance with an embodiment is shown in FIG. 6D. The sculptor-designed 3D model of the enriched binder is shown in the center. The binder is shown in blue and target structure show in white. Each scatter plot shows a yeast surface display experiment analyzed with fluorescence assisted cell sorting (FACS). Display signal is plotted on the x-axis and binding signal on the y-axis. FACS gates are shown in red and annotated with binding population percentage. The two plots in the upper left correspond to binding profiles of the negative control and design target, T3. All other plots show binding profiles of mutants that attenuated binding relative to the native target. Attenuating mutants are mapped onto the target surface and colored magenta.

To perform epitope mapping, mutations can be added to the designed interface. Several mutations may affect binding (FIG. 6D). Two mutations, F7A and F30A may reduce binding and two other mutations, R34V and R37V, may abolish binding almost completely. These data suggest that the binder contacts the region comprised of F7, F30, R34, and R37, which is colored magenta on the designed complex in FIG. 6C. The result shows a significant overlap between these mutations and the intended epitope, providing evidence in agreement with the design model.

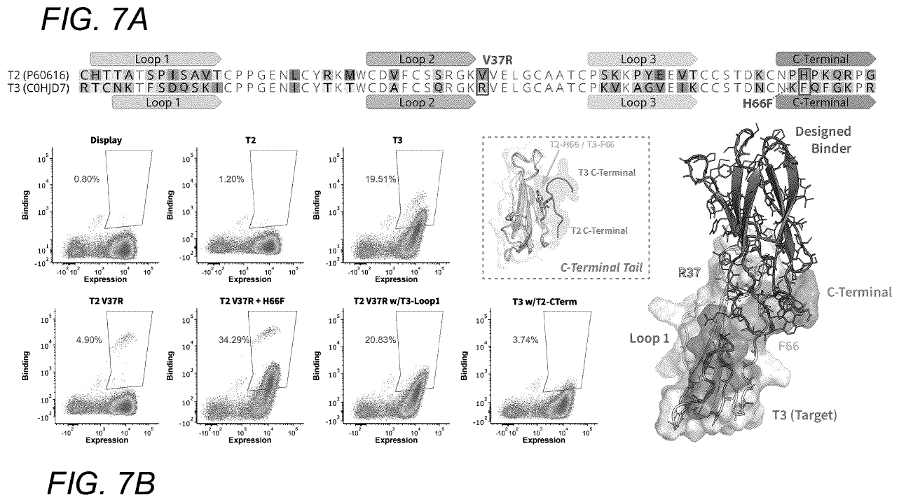

To further confirm the binder's epitope and understand cross-toxin specificity, several embodiments identify the set of features in T3 required to rescue binding in T2. From sequence alignments, two residues, R37 and F66 can be conserved across T3/4/5, but not in T1/2. While experiments in FIG. 6D suggest that R37 may be essential to T3 binding, the equivalent mutation in T2 alone may not be sufficient to rescue binding (FIG. 7, T2 V37R). However, combining the V37R mutation with H66F may allow for recovery of binding (FIG. 7, T2 V37R+H66F). From the crystal structure data, residue F66 residue appears to play a crucial role in determining the trajectory of the C-terminal tail (FIG. 7B), and the rescue-effect suggests that the binder is contacting F66 and likely other regions of tail as well. The importance of the C-terminal tail is further supported by the loss in binding when replacing the tail of T3 with that of T2 (FIG. 7, T3 w/T2-CTerm). V37R and H66F may be two of only three residues in that differ in the user-specified epitope between T3/4/5 and T1/2 (FIG. 6A, arrows), further suggesting that this feature is likely the main determinant of binder specificity in the native targets. T2 binding may be rescued without the H66F mutation by replacing the first loop of T2 with that of T3, in combination with the V37R mutation (FIG. 6, T2 w/T3-Loop1).

In many embodiments, the binder's epitope agrees closely with the design model and lies within the user-specified epitope. The T3 mutation data confirms that mutations at the interface result in loss of binding, and the T2 rescue data support this, suggesting that specificity to T3/5/6 is likely due to the combination of the R37 and F66 residues. The rescue experiments further suggest the binder contacts Loop 1, again in agreement with the T3 and the design model. While it is likely necessary to affinity mature the Sculptor-designed binder for practical use, the total library size used for these experiments is on the order of about 103 and considerably smaller than that required by conventional yeast display libraries. Sculptor in accordance with many embodiments provide a powerful way to build small naïve libraries for obtaining initial binders for evolution, and a significant step towards broadly neutralizing binder design.

### Design an Antibody

Modifications to the template 3D backbone, interaction field, and target binding site can be adapted to generate binding molecules for any arbitrary binding targets. Sculptor can be adapted for antibody designs. Dynamic loop assignment can be incorporated in antibody designs. The complementary determining regions (CDRs) of antibodies play an important role in antigen recognition. In general, the CDR loops in the heavy chain are more frequently involved in antigen binding than those in the light chain. Dynamic loop assignment occurs with each loop of the algorithm. At the start of each loop, the residues are changed in order to interact with the target epitope. Various methods such as a neural network classifier can be used to predict which residues are CDR loop residues. Once the CDR residues are identified, the interactions are restricted to be only with those residues, such that the antibody binders interact with the target epitope only via the CDR loops and not via other regions in the protein.

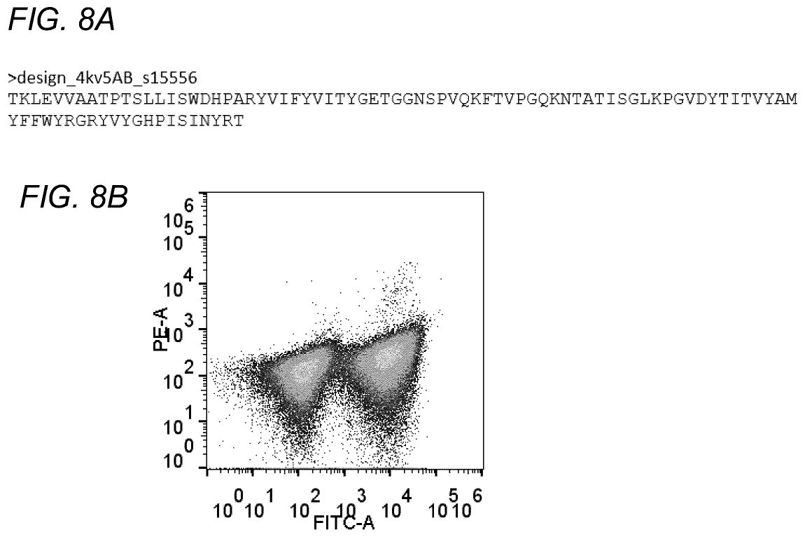

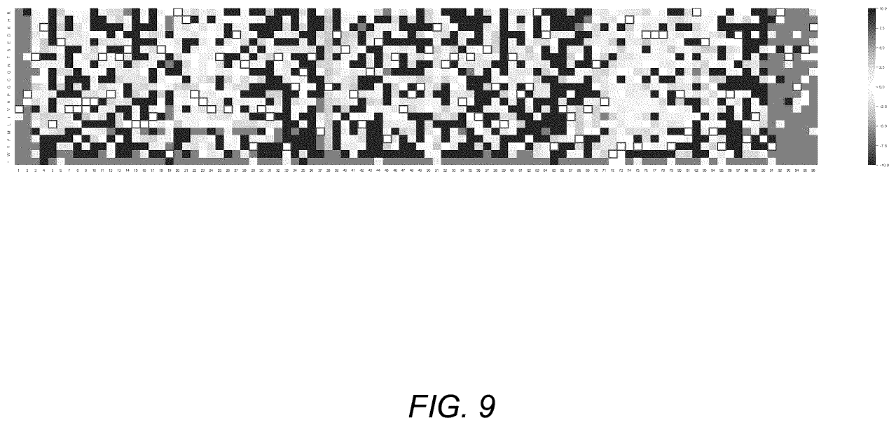

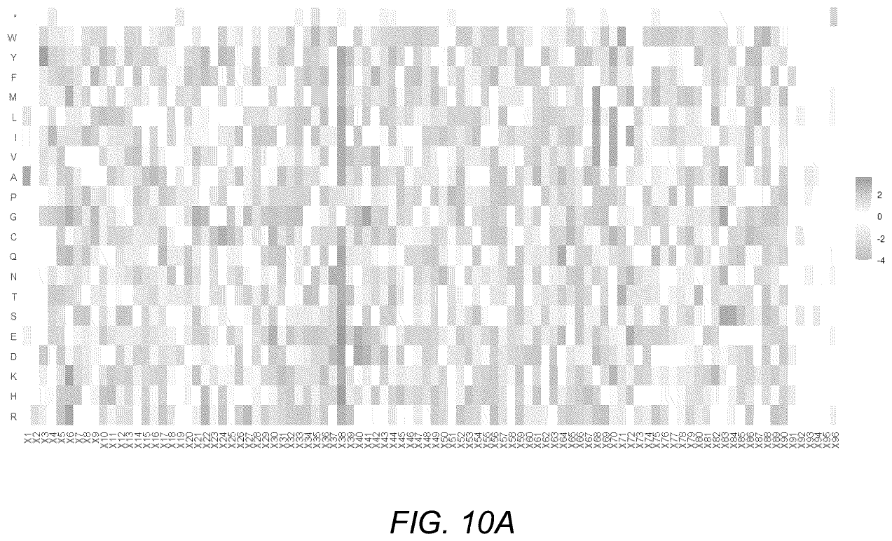

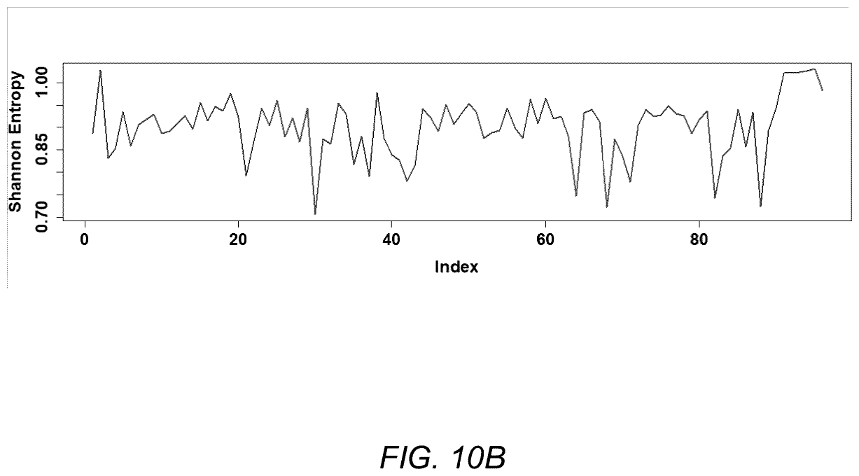

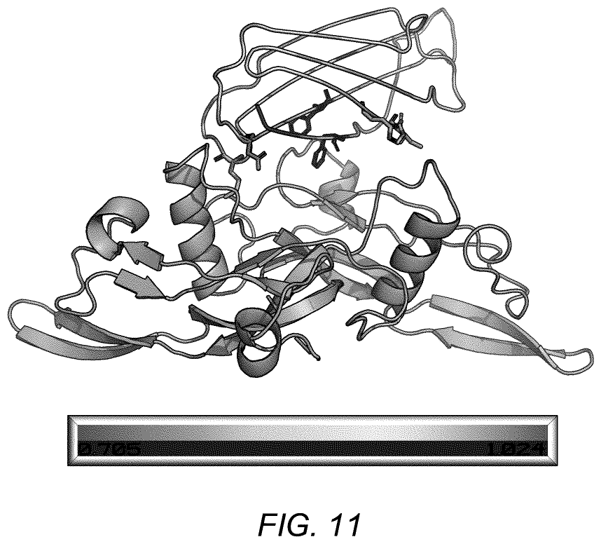

Transforming growth factor beta 1 or TGF-beta1 or TGF-β1 is a polypeptide member of the transforming growth factor beta superfamily of cytokines that controls proliferation, differentiation, and other functions in many cell types. Various embodiments use Sculptor to design TGF-beta1 binders. FIG. 8A illustrates sequence for binders against TGF-beta1 in accordance with an embodiment. FIG. 8B illustrates characterization of the interface in accordance with an embodiment. Site-saturated mutagenesis can be used to characterize the interface. TGF-beta1 is labeled at about 0.95 nM. FIG. 9 illustrates a heatmap of the result relative to wildtype enrichment in accordance with an embodiment. FIG. 10A and FIG. 10B illustrate Shannon entropy in accordance with an embodiment. Shannon entropy shown in FIGS. 10A and 10B convert the information in FIG. 9 into per-position Shannon entropy. FIGS. 9 and 10 are similar but with a different scaling. FIG. 11 illustrates a protein structure based on the entropy calculation in accordance with an embodiment. Mapping the entropy to design model reveals that the immutable positions match the interface: low entropy residues in the interface are shown as sticks, colored by the entropy values.

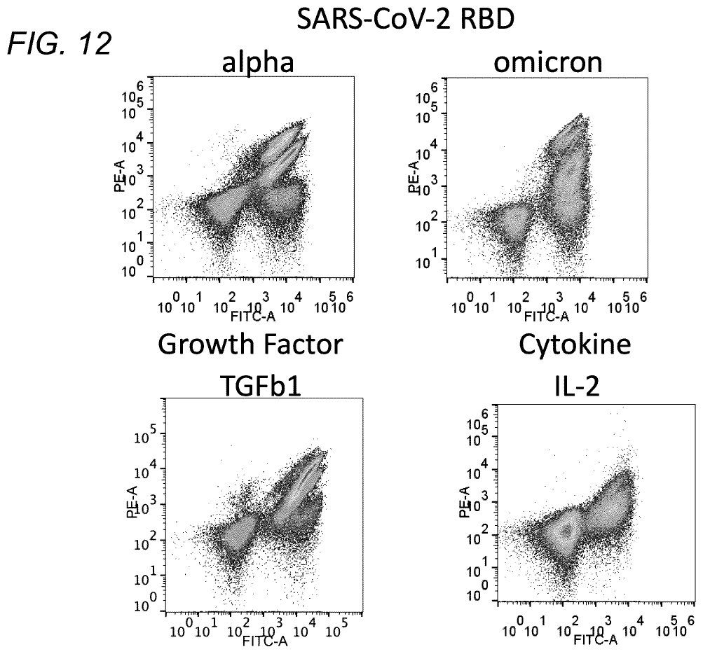

FIG. 12 illustrate a yeast surface display experiment of various binders analyzed with fluorescence assisted cell sorting (FACS) in accordance with an embodiment. The binders shown in FIG. 12 include SARS-COV-2 alpha variant, SARS-CoV-2 omicron variant, growth factor TGF-beta1, and cytokine IL-2.

### Exemplary Embodiments

The following section provides specific examples of the Sculptor processes to determine compositions and structures for proteins. It will be understood that the specific embodiments are provided for exemplary purposes and are not limiting to the overall scope of the disclosure, which must be considered in light of the entire specification, figures and claims.

### Data Selection and Processing

Monobody structures that are similar in length (approx 91 residues) can be manually chosen from PDB, yielding the following list of 34 structures: 6b2bC, 710gH, 5dc0A, 4jegB, 5e95B, 5g15B, 3csbA, 5dc4B, 6tlcD, 3csgA, 7jw7B, 6bqoC, 2obgA, 6o02B, 5kbnD, 6b2aD, 6apxB, 5a43D, 5ecjE, 7l0fB, 3qhtD, 5komD, 5dc9B, 6bynM, 5mtmB, 2ocfD, 3uyoD, 5n7eA, 5v7pD, 5mtjB, 4je4B, 7jxuD, 5mtnB, 6bx5D. Next, each template is structurally padded or truncated to 91 residues. 12 triple mutants can be generated randomly per structure using PyRosetta, with each mutation localized to the loop regions of the monobodies. Including the native PDBs, this yielded a total of 442 seed structures for simulation.

Data Generation using Molecular Dynamics

To generate training data, molecular dynamics simulations can be used. All simulations are performed using the AMBER03 force field for the proteins, in a dodecahedron solvated box using explicit TIP3P solvent model. The solvated structures are energy minimized with a steepest descent algorithm using Gromacs 2020, until the maximum force is below 100 KJ mol⁻¹ nm⁻¹. A step size of 0.01 nm and a cut-off distance of 1.2 nm for the neighbor list, Coulomb interactions and van der Waals interactions are used during the energy minimization. Following energy minimization, structures are equilibrated or 1 ns, and all bonds are constrained with the LINCS algorithm. Virtual sites are used to enable an integration time step of 4 fs. A cut-off distance of 1.1 nm is used for the neighbor list, and a cut-off distance of 0.9 nm is used for Coulomb and van der Waals interactions. The Verlet cut-off scheme is used for the neighbor list. For the long-range electrostatic interactions, the particle mesh Ewald method can be used with a Fourier spacing of 0.12 nm. Throughout the simulations, stochastic velocity rescaling (v-rescale) thermostat is used to maintain the temperature of the system at 300 K.

Starting from the equilibrated structure for each of 442 monobodies, unbiased MD simulations are performed on the Folding@home distributed computing project. Ten parallel simulations, each starting with randomly generated initial atomic velocities, are initiated from the equilibrated structures. An integration time step of 4 fs is used during these Folding@home simulations. The temperature of the system is maintained at 300 K using V-rescale thermostat and the pressure is controlled at 1 bar using Parrinello-Rahman barostat. Atomic coordinates of the protein are saved at every 20 ps time step in the Folding@home output trajectories, and an aggregate simulation time of more than 300 ns is collected for each monobody structure (164 s aggregate simulation). Folding@home output trajectories are further subsampled to retain every 5th frame in the trajectory. The resulting 1.6M structures are used as training examples for the VAE. To create a dataset for the sequence design module, we randomly selected 40,000 of the clustered frames from the MD simulations and extracted the 3.64M residue environments corresponding to each residue in each frame. For the test set of the sequence design module, 100 residue environments per amino acid type can be randomly selected to ensure class balancing.

### Model Architectures and Training

The VAE architecture is reported with input and output dimensions adjusted to match the size of the structurally padded monobodies. In addition to the backbone atoms, the current model also includes Cb atoms, which are artificially placed for residues lacking them. Methods and scripts for structural padding are also provided in the IgVAE reference. The architecture of the sequence design module processes backbone atom coordinates (N, Ca, C, O), and Cb. This model also may not construct side-chains, and perform classification at each position, independent of amino acid identities at other positions. The residue environments used as inputs are 8 Å voxelized cubes centered at the residue-of-interest and with a voxel size of 0.5 Å. All models are trained on RTX 2080Ti's using the PyTorch deep learning framework.

### Sequence Design

Sequence design is performed in two phases. In the first, a list of interacting residues is created using nearest alignments to the interaction field and merged with the list of design-module-proposed amino acid identities. Any amino acids with greater than 5% probability under the classifier are included as candidates. The combined list is passed to Rosetta for joint sequence design at all positions. During this phase a cartesian relax is performed during design, allowing for optimization of bond lengths and angles. In the second phase, Rosetta is used to optimize the binding interface by designing without restrictions to amino acid identity, and the designed structure undergoes an unconstrained relax. The first phase uses the PyRosetta interface, while the second uses a RosettaScripts interface.

### Interaction Field Construction

Protein structures are downloaded from RCSB Protein Data Bank with 40% sequence homology, X-ray diffraction resolution≤2.0, and Robs≤0.2. This results in a list of 14,680 structures. Each structure is analyzed using Arpeggio to identify all residues involved in polar interactions with interaction distances 5 Å. Interacting residues on the same PDB chain are required to be more than 6 residues apart. The interacting residues are grouped by amino acid identity resulting in 210 groups (e.g. Ala-Ala, Ala-Arg, . . . ). For each group an all-vs-all distance matrix is computed using RMSD. The backbone atoms (N, C, Ca, O) and Cb atom is used for the RMSD calculations. For glycine a virtual Cb atom was generated. In groups where both interacting amino acids are the same identity (e.g. Ala-Ala, Arg-Arg, . . . ) RMSD is computed twice by flipping residue order, with the minimum of the two being used as the final value. Clusters are created using hierarchical clustering on the distance matrix with a distance threshold of 0.3 Å dRMSD and ward linkage to minimize variance within each cluster. For each cluster, the representative pair can be determined by the pair that minimizes the sum of the distances to all other pairs in the same cluster

### Recombinant Production of Three Finger Toxins

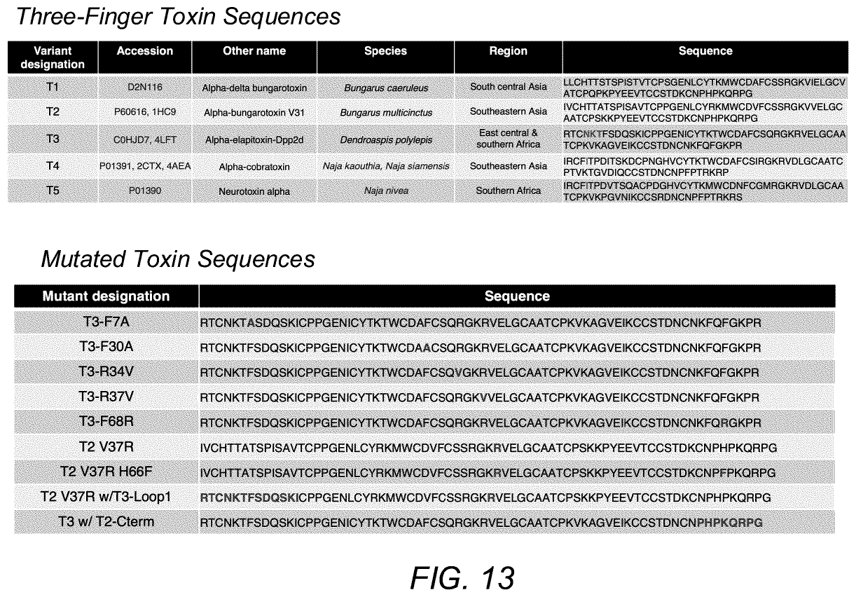

Long chain alpha-neurotoxin three finger toxin (3FTX) variant sequences (FIG. 13) from various snake species are selected from NCBI databases and cloned into a mammalian expression vector containing a C-terminal Avi™ tag followed by a WELQut™ cleavage tag and a rabbit Fc tag by Genscript. Plasmid DNA is prepped and co-transfected with a plasmid expressing the BirA enzyme for in vivo biotinylation into Expi293 cells using FectoPRO®. Cells are grown for 5 days shaking at 240 RPM, 37° C. with 8% CO2 in Expi293™ expression medium. Approximately 24 hours post-transfection, cells are stimulated with 3 mM valproic acid, 1.8 mg/ml D-glucose, and 20 nM d-biotin. Supernatant is harvested from cell cultures and incubated overnight rotating at 4° C. with rProtein A Sepharose affinity resin. The affinity resin is washed once with 500 mM NaCl, 1×PBS and twice with 1×PBS before transferring to 20 mM Tris, 150 mM NaCl, pH 7.4. WELQut protease is added to the resin and incubated with rotation for 3 hours at 30° C. Supernatant is harvested from the resin and incubated with HisPur™ Ni-NTA resin for 30 minutes at room temperature to remove the His-tagged protease, followed by a second incubation with Protein A resin for 30 minutes to remove any residual Fc tag. The final supernatant is concentrated to >1 mg/ml using a 3 kDa centrifugal filter unit and frozen in aliquots stored at −80° C. Biotinylation of the purified 3FTX was confirmed using the Pierce™ biotin quantitation kit. Tetramers of streptavidin conjugated to biotinylated 3FTX are formed via 60 min incubation of 4:1 3FTX: streptavidin-APC at room temperature prior to use.

### Yeast Library Production and Transformation

Sculptor is used to generate 12,000 monobody designs against the common epitope for each of the target structures, PDB:1HC9 (A), PDB:4LFT (B), and PDB:2CTX (A). The top 3,000 designs for each target are selected based on a combined ranking over Rosetta ddG, Sc, and buried SASA. Selected sequences are codon optimized for expressing in Saccharomyces cerevisiae and synthesized by Twist Biosciences. The pooled monobody libraries are PCR amplified and extended with long oligos to include 80 bp of upstream homology and 55 bp of downstream homology with the pYDSI2u surface display vector. The amplified monobody encoding DNA and linearized pYDSI2u vector are transformed into yeast for homologous recombination.

### Fluorescence-Activated Cell Sorting

In vitro engineering of antibodies or nanobodies can lead to constructs that are polyspecific, so both positive and negative selections are employed to enhance the specificity of the synthetic constructs. Yeast cells are alternatively selected as 3FTX binders (affinity sorts), or as poly-specificity reagent (PSR) non-binders (negative sorts). During affinity sorting, 1-5*107 induced yeast cells are incubated for 60 min rotating at 4° C. with streptavidin-APC tetramerized 3FTX in PBSA (PBS containing 1% BSA) supplemented with 1 μg/mL anti-V5-AF405 to check the yeast display. During PSR sorting, induced yeast cells are incubated for 60 min rotating at 4° C. with biotinylated HEK-cell soluble membrane protein extracts in PBSA, washed with PBSA, then coupled to 1 μg/mL of both fluorophores (streptavidin-APC and anti-V5-AF405) for 20 min. Yeast cells are then washed once and resuspended in PBSA for sorting on a FACS Melody. Selected yeast cells are sorted into SD-Ura medium, grown shaking overnight at 30° C. and induced for consecutive rounds of selection. Induction medium contains 20 g/L galactose, 1 g/L glucose, 6.7 g/L yeast nitrogen base, 5 g/L bacto-casamino acids, 38 mM disodium phosphate, 72 mM monosodium phosphate, and 419 UM L-tryptophan. For the 1st, 2nd and 3rd affinity sorts, 500, 500, and 20 nM of streptavidin-3FTX_LCa-5 tetramer was used, respectively. For the single PSR sort (between the 2nd and 3rd affinity sorts) 20 μg/ml of biotinylated CHO-cell soluble membrane protein extract is used.

### Monobody Sequencing and Analysis

Serial dilutions of the final affinity sort are plated on SD-Ura agar. After 3 days at 30° C., 30 single yeast colonies are combined and inoculated into 2 mL SD-Ura medium, and grown overnight shaking at 30° C. The DNA from the yeast cells is miniprepped in the presence of zymolyase and transformed into DH10B competent E. coli. 30 single colonies are picked and analyzed via Sanger sequencing. The same yeast culture is also analyzed via flow cytometry for binding to various 3FTX variants and mutants] using 100 nM of streptavidin-APC tetramerized 3FTX.

### DOCTRINE OF EQUIVALENTS

As can be inferred from the above discussion, the above-mentioned concepts can be implemented in a variety of arrangements in accordance with embodiments of the invention. Accordingly, although the present invention has been described in certain specific aspects, many additional modifications and variations would be apparent to those skilled in the art. It is therefore to be understood that the present invention may be practiced otherwise than specifically described. Thus, embodiments of the present invention should be considered in all respects as illustrative and not restrictive.

As used herein, the singular terms “a,” “an,” and “the” may include plural referents unless the context clearly dictates otherwise. Reference to an object in the singular is not intended to mean “one and only one” unless explicitly so stated, but rather “one or more.”

As used herein, the terms “approximately,” and “about” are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

What is claimed is:

1. A method of synthesizing a binding protein comprising:
identifying a target structure;
constructing at least one interaction field describing physical interaction properties of atoms in the target structure;
optimizing a binding protein to the target structure:
generating a candidate binding protein using a generative model;
fitting the candidate binding protein and target structure in a virtual space according to a homogenous transformation function;
using a loss function to produce an error value which evaluates a binding affinity between the candidate binding protein and the target structure based on the at least one interaction field; and
while the error value is above a threshold value, providing the error value to the generative model and to the homogenous transformation function in order to inform the generation of a subsequent candidate binding protein and fitting to lower the error value;
outputting the binding protein until the error value reaches a stopping threshold; and
synthesizing the binding protein.

2. The method of claim 1, wherein the target structure is selected from the group consisting of: a protein, a region of a protein, an epitope, an antibody, a polypeptide, a region of a polypeptide, a nucleic acid, a DNA, an RNA, a sugar molecule, a monosaccharide, a disaccharide, a polysaccharide, and a small molecule.

3. The method of claim 1, wherein the at least one interaction field comprises interactions selected from the group consisting of: Coulomb interactions, hydrogen bonds, π-π interactions, cation-π interactions, van der Waals interactions, and a virtual constraint.

4. The method of claim 1, further comprising providing the generative model with a template backbone structure based on the target structure, wherein the template backbone structure is selected from the group consisting of: a monobody, an antibody, a nanobody, a single-chain variable fragment, a designed ankyrin repeat protein, and a lectin.

5. The method of claim 4, wherein the generative model iteratively modifies the template backbone structure.

6. The method of claim 1, further comprising generating amino acid sequences of the binding protein.

7. The method of claim 1, further comprising ranking a set of the subsequent candidate binding proteins based on their binding affinity to the target structure.

8. The method of claim 1, wherein the synthesized binding protein is configured to be used in prokaryotes or eukaryotes.

9. The method of claim 1, wherein the synthesized binding protein is configured to be used in in vitro or in vivo assays.

10. A method of synthesizing a binding protein comprising,
identifying a target structure;
generating at least one interaction field describing physical interaction properties of atoms in the target structure;
generating a candidate binding protein using a generative model;
fitting the candidate binding protein and the target structure in a virtual space according to a homogenous transformation function;
using a loss function to produce an error value which evaluates a binding affinity between the candidate binding protein and the target structure based on the at least one interaction field;
providing the error value to the generative model and to the homogenous transformation function;
generating a subsequent candidate binding protein using the generative model and the error value;
fitting the subsequent candidate binding protein and the target structure according to the homogenous transformation function and the error value; and
synthesizing the subsequent candidate binding protein as the binding protein.

11. The method of claim 10, wherein the target structure is selected from the group consisting of: a protein, a region of a protein, an epitope, an antibody, a polypeptide, a region of a polypeptide, a nucleic acid, a DNA, an RNA, a sugar molecule, a monosaccharide, a disaccharide, a polysaccharide, and a small molecule.

12. The method of claim 10, wherein the at least one interaction field comprises interactions selected from the group consisting of: Coulomb interactions, hydrogen bonds, π-π interactions, cation-π interactions, van der Waals interactions, and a virtual constraint.

13. The method of claim 10, further comprising providing the generative model with a template backbone structure based on the target structure, wherein the template backbone structure is selected from the group consisting of: a monobody, an antibody, a nanobody, a single-chain variable fragment, a designed ankyrin repeat protein, and a lectin.

14. The method of claim 13, wherein the generative model iteratively modifies the template backbone structure.

15. The method of claim 10, further comprising generating amino acid sequences of the subsequent candidate binding protein.

16. The method of claim 10, further comprising ranking a set of the subsequent candidate binding proteins based on their binding affinity to the target structure.

17. The method of claim 10, wherein the synthesized binding protein is configured to be used in prokaryotes or eukaryotes.

18. The method of claim 10, wherein the synthesized binding protein is configured to be used in in vitro or in vivo assays.

19. A method for generating a binding molecule, comprising:
identifying a target structure having a target binding site;
generating at least one interaction field describing physical interaction properties of atoms in the target binding site;
using a generative model to create a 3D model of a candidate binding molecule;
fitting the candidate binding molecule to the target binding site using a homogenous transformation function based on the at least one interaction field in a virtual space containing the 3D model of the candidate binding molecule and the target structure;
calculating an error in the fitting using a loss function; and
refining the candidate binding molecule and the homogenous transformation using the error until a stopping threshold is reached.

20. The method of claim 19, wherein the stopping threshold is a predetermined number of iterations.

21. The method of claim 19, wherein the stopping threshold is a minimum acceptable error value.

22. The method of claim 19, wherein the stopping threshold is a minimum change in error value required to continue the refining.

23. The method of claim 19, wherein the target binding site is on a surface of the target structure.

24. The method of claim 19, wherein the loss function further determines a number of residues allowed to overlap the interaction field.

25. The method of claim 19, wherein the target structure is selected from the group consisting of: a protein, a region of a protein, an epitope, an antibody, a polypeptide, a region of a polypeptide, a nucleic acid, a DNA, an RNA, a sugar molecule, a monosaccharide, a disaccharide, a polysaccharide, and a small molecule.

26. The method of claim 19, wherein the at least one interaction field comprises interactions selected from the group consisting of: Coulomb interactions, hydrogen bonds, π-π interactions, cation-π interactions, van der Waals interactions, and a virtual constraint.

27. The method of claim 19, wherein the 3D model is selected from the group consisting of: a monobody, an antibody, a nanobody, a single-chain variable fragment, a designed ankyrin repeat protein, and a lectin.

28. The method of claim 27, wherein the generative model iteratively modifies the 3D model.

29. The method of claim 19, further comprising ranking a set of the candidate binding molecules based on their binding affinity to the target structure.
